(19) United States
(12) Patent Application Publication
(10) Pub. No.: US 2022/0370296 A1
(43) Pub. Date: Nov. 24, 2022

(54) NASOGASTRIC TUBE CONNECTOR DEVICE FOR RESPIRATOR MASK

(71) Applicant: HSINER CO., LTD., Taichung City (TW)

(72) Inventor: Eric CHANG, Taichung City (TW)

(73) Assignee: HSINER CO., LTD., Taichung City (TW)

(21) Appl. No.: 17/398,281

(22) Filed: Aug. 10, 2021

(30) Foreign Application Priority Data
May 20, 2021 (TW) 110118313

(51) Int. Cl. A61J 15/00; A61M 16/06

(52) U.S. Cl. CPC A61J 15/0061; A61M 16/06; A61J 15/0003

(57) ABSTRACT

A nasogastric tube connector device includes an annular connector body for detachably engaging a through hole of a respirator mask having an inner peripheral surface defining a space, and a fixed member having a first concave surface and two side surfaces cooperating with the inner peripheral surface to define an opening of the space for passing of a nasogastric tube therethrough. A first plug member is detachably inserted through the opening into the space, and includes a second concave surface cooperating with the first concave surface to define a main tube hole communicating with the space. A second plug member is detachably inserted into the main tube hole to sealingly close the opening.

### CROSS-REFERENCE TO RELATED APPLICATION

This application claims priority to Taiwanese Patent Application No. 110118313, filed on May 20, 2021.

### FIELD

The disclosure relates to a nasogastric tube connector device for a respirator mask.

### BACKGROUND

A cushion mounted on a respirator mask is disclosed in U.S. patent Ser. No. 10/722,672B2. The respirator mask is connected to an oxygen supply tube. The cushion has a face contacting portion for contacting a face of a user and for surrounding a nose or mouth of the user, and at least one thin region located on the face contacting portion. The at least one thin region has a material softer than that of the cushion. A nasogastric tube can be inserted between the at least one thin region and the user's face, and is fixed to the user's nose for feeding. However, when the nasogastric tube is inserted into the respirator mask, a gap still exists between the at least one thin region and the user's face, which reduces the airtightness of the respirator mask, resulting in poor oxygen supply.

### SUMMARY

Therefore, an object of the present disclosure is to provide a nasogastric tube connector device that can optionally mount a nasogastric tube on a respirator mask and that can provide a good airtight effect.

Accordingly, a nasogastric tube connector device of this disclosure is suitable for connection with a respirator mask having a through hole. The nasogastric tube connector device includes an annular connector body for detachably engaging the through hole, a first plug member and a second plug member. The annular connector body includes an inner peripheral surface defining a space, and a fixed member connected to the inner peripheral surface and having a first concave surface, and two side surfaces respectively extending from two opposite ends of the first concave surface toward and connected to the inner peripheral surface. The side surfaces, the first concave surface and the inner peripheral surface cooperatively define an opening of the space for passing of a nasogastric tube therethrough.

The first plug member is detachably inserted through the opening into the space, and includes a second concave surface cooperating with the first concave surface to define a main tube hole communicating with the space for insertion of the nasogastric tube therethrough. The second plug member is detachably inserted into the main tube hole to sealingly close the opening of the space.

### BRIEF DESCRIPTION OF THE DRAWINGS

Other features and advantages of the disclosure will become apparent in the following detailed description of the embodiment with reference to the accompanying drawings, of which:

FIG. 1 is an exploded perspective view of a nasogastric tube connector device according to an embodiment of the present disclosure and a respirator mask;

FIG. 2 is an assembled perspective view of the embodiment and the respirator mask with the embodiment in a use state;

FIG. 3 is a perspective view of the embodiment in an open state;

FIG. 4 is a bottom view of the embodiment in the open state;

FIG. 5 is a top view of the embodiment in the open state;

FIG. 6 is a view similar to FIG. 2, but with the embodiment placed in the open state;

FIG. 7 is a top view of the embodiment in the use state;

FIG. 8 is a sectional view of the embodiment taken along line VIII-VIII of FIG. 7;

FIG. 9 is a view similar to FIG. 8, but with the embodiment in a closed state; and

FIG. 10 is an assembled perspective view of the embodiment and the respirator mask with the embodiment in the closed state.

### DETAILED DESCRIPTION

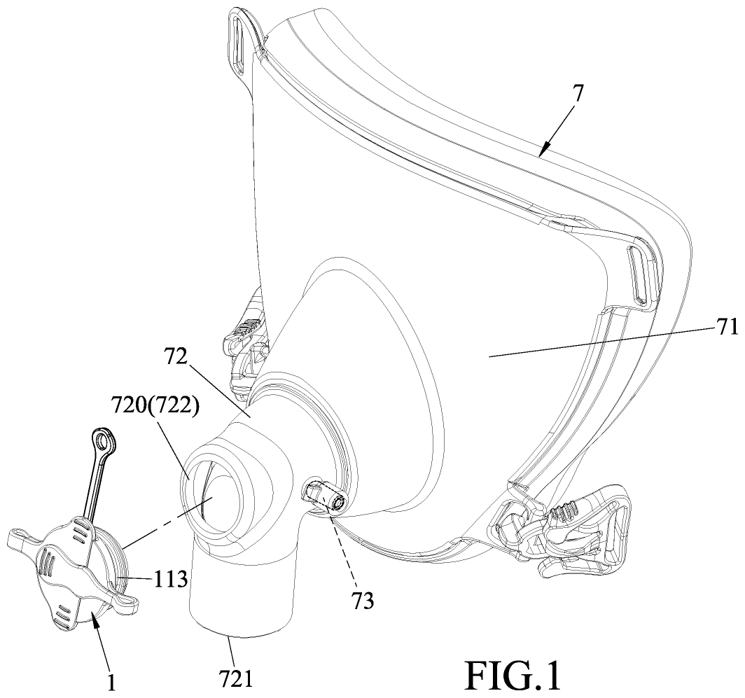

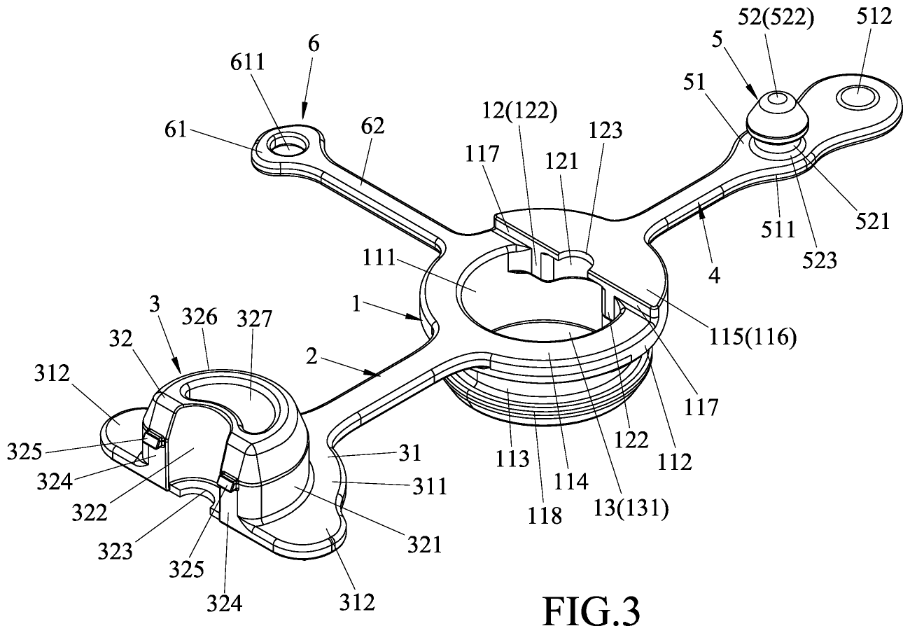

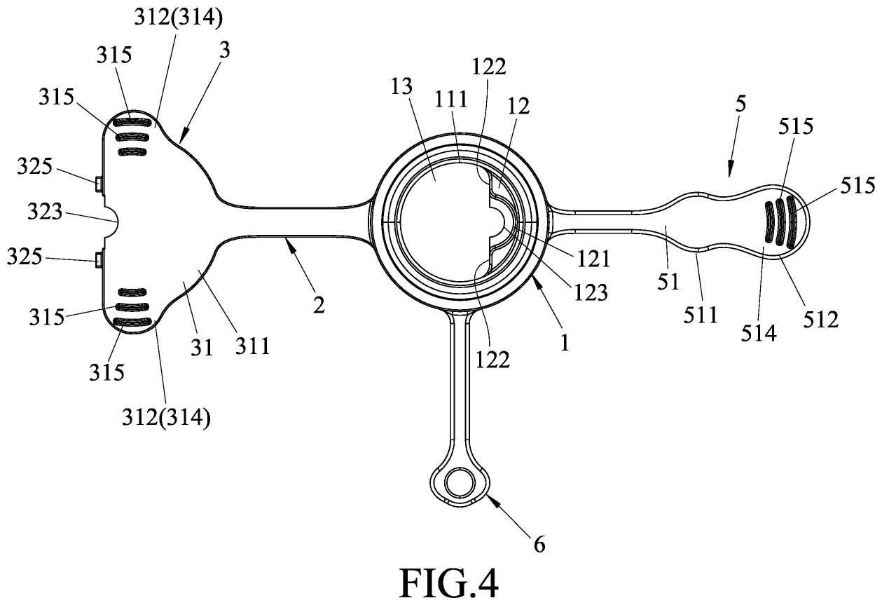

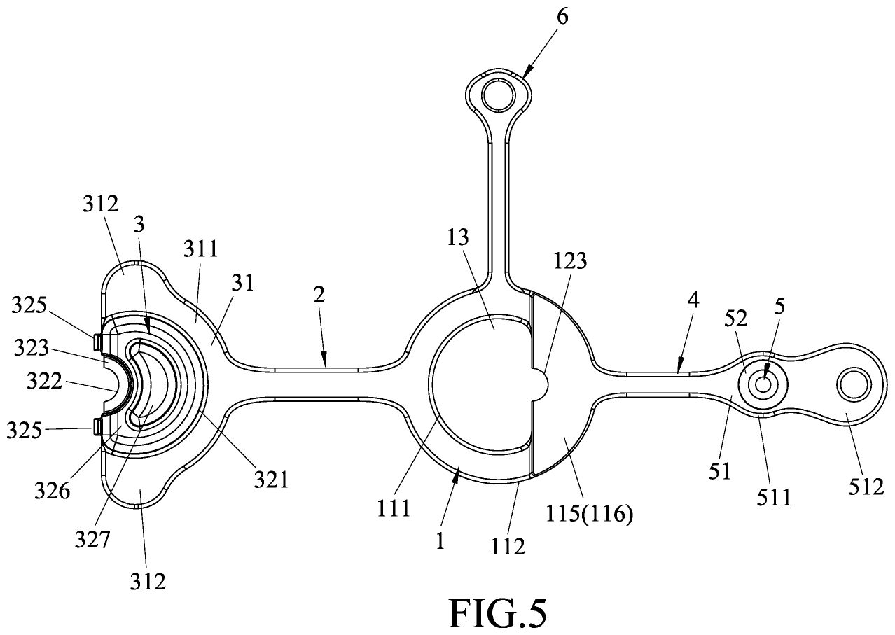

Referring to FIGS. 1 to 5, a nasogastric tube connector device according to an embodiment of the present disclosure is suitable for connection with a respirator mask 7. The respirator mask 7 includes a mask body 71, a breathing tube 72 connected to the mask body 71, and an oxygen supply tube 73 connected to the breathing tube 72. The breathing tube 72 has an air inlet 721, and a through hole 722 communicating with the air inlet 721 and defined by a hole-defining wall 720. The nasogastric tube connector device of this disclosure is integrally formed of elastic silicone, and includes an annular connector body 1, a first connecting tab 2, a first plug member 3, a second connecting tab 4, a second plug member 5, and a retaining member 6.

The annular connector body 1 is detachably engaged with the through hole 722, and includes an inner peripheral surface 111 defining a space 13, an outer peripheral surface 112 opposite to the inner peripheral surface 111, an annular groove 113 extending inwardly from the outer peripheral surface 112, an upper peripheral surface 114 connected between upper ends of the inner and outer peripheral surfaces 111, 112, a protruding platform 115 protruding upwardly from the upper peripheral surface 114 and having a substantially semicircular shape, and an annular protrusion 118 that is formed on the outer peripheral surface 112, that is adjacent to the annular groove 113 and that is distal to the upper peripheral surface 114. The protruding platform 115 has a platform surface 116 opposite to the upper peripheral surface 114, and two abutment surfaces 117 extending transversely and respectively from two opposite sides of the platform surface 116 toward the upper peripheral surface 114.

A fixed member 12 is connected to the inner peripheral surface 111 and a bottom portion of the protruding platform 115, and has a first concave surface 121 proximate to the upper peripheral surface 114, two side surfaces 122 respectively extending from two opposite ends of the first concave surface 121 toward and connected to the inner peripheral surface 111, and a first concave flange 123 extending inwardly and radially from an upper end of the first concave surface 121 and connected to the platform surface 116. The side surfaces 122 are further respectively connected to the abutment surfaces 117. In this embodiment, the first concave surface 121 has a semicircular shape. The first concave surface 121, the side surfaces 122, and the inner peripheral surface 111 cooperatively define an opening 131 of the space 13 for passing of a nasogastric tube 8 therethrough.

The first connecting tab 2 interconnects the connector body 1 and the first plug member 3, and extends from a junction of the outer peripheral surface 112 and the upper peripheral surface 114 to the first plug member 3.

The first plug member 3 includes a first plug body 32 detachably inserted through the opening 131 into the space 13, and a first extension portion 31 extending outwardly from the first plug body 32 and connected to the first connecting tab 2.

The first plug body 32 defines a buffer chamber 327 having a closed upper end and an open lower end, and includes a convex surface 321, a second concave surface 322 opposite to the convex surface 321, a second concave flange 323 extending inwardly and radially from an upper end of the second concave surface 322, two connecting surfaces 324 each of which extends from one of two opposite ends of the second concave surface 322 to a respective one of two opposite ends of the convex surface 321, two protrusions 325 respectively protruding from and respectively inclined with respect to the connecting surfaces 324, and an end surface 326 connected to lower ends of the convex surface 321, the second concave surface 322 and the connecting surfaces 324.

The buffer chamber 327 is cooperatively defined by the convex surface 321, the second concave surface 322, the connecting surfaces 324 and the end surface 326. The open lower end of the buffer chamber 327 is formed in the end surface 326. The convex surface 321 has a curvature matching that of the inner peripheral surface 111 of the connector body 1. In this embodiment, each of the second concave surface 322 and the convex surface 321 has a semicircular shape. The second cover section 511, the neck portion 521 and the head portion 522 cooperatively define an annular engaging groove 523.

The second concave surface 322 cooperates with the first concave surface 121 to define a main tube hole 14 communicating with the space 13 for insertion of the nasogastric tube 8 therethrough. The second concave flange 323 cooperates with the first concave flange 123 to define an auxiliary tube hole 15 communicating with the main tube hole 14 and having a hole diameter smaller than that of the main tube hole 14. In this embodiment, the hole diameter of the main tube hole 14 is 8 mm, while the hole diameter of the auxiliary tube hole 15 is 6 mm, but is not limited thereto. Each of the first and second concave flanges 123, 323 is made of an elastic material, such as silicone rubber.

The first extension portion 31 has a first cover section 311 connected to the first connecting tab 2 and an upper periphery of the convex surface 321 of the first plug body 32, and two first holding sections 312 extending outwardly and respectively from two opposite sides of the first cover section 311. Each first holding section 312 has a plurality of protruding ribs 315 formed on an outer surface 314 thereof. In other variations of this embodiment, the number of the first holding sections 312 may be one or more than two, but is not limited thereto.

The second connecting tab 4 interconnects the connector body 1 and the second plug member 5, and extends from a junction of the outer peripheral surface 112 and the platform surface 116 to the second plug member 5.

The second plug member 5 includes a second plug body 52 for detachable insertion into the main tube hole 14 through the auxiliary tube hole 15 to sealingly close the opening 131 of the space 13, and a second extension portion 51 extending outwardly from the second plug body 52 and connected to the second connecting tab 4. The second plug body 52 has a neck portion 521, and a head portion 522 connected to and gradually tapering away from the neck portion 521.

The second extension portion 51 includes a second cover section 511 connected to the second connecting tab 4 and the neck portion 51, and a second holding section 512 extending outwardly from the second cover section 511 in a direction away from the second connecting tab 4 and having a plurality of protruding strips 515 formed on an outer surface 514 thereof. The second cover section 511, the neck portion 521 and the head portion 522 cooperatively define an annular engaging groove 523 therebetween.

The retaining member 6 is connected to the connector body 1, and is located between the first and second connecting tabs 2, 4. The retaining member 6 includes a connecting portion 62 extending from the upper peripheral surface 114 away from the connector body 1, and a ring portion 61 connected to the connecting portion 62 opposite to the connector body 1 and sleeved on the oxygen supply tube 73 to prevent the same from separating from the breathing tube 72. The ring portion 61 has a retaining hole 611 for extension of the oxygen supply tube 73 therethrough.

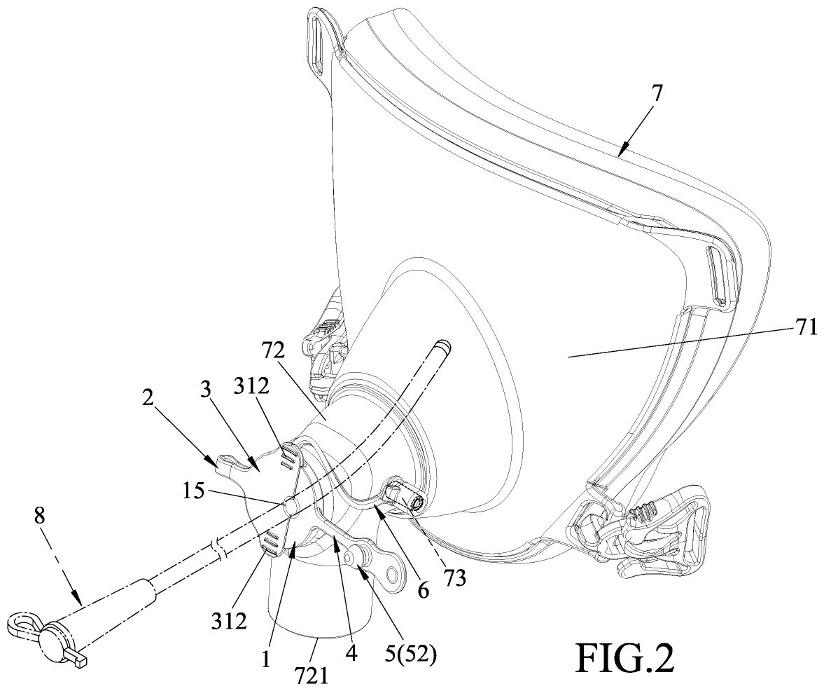

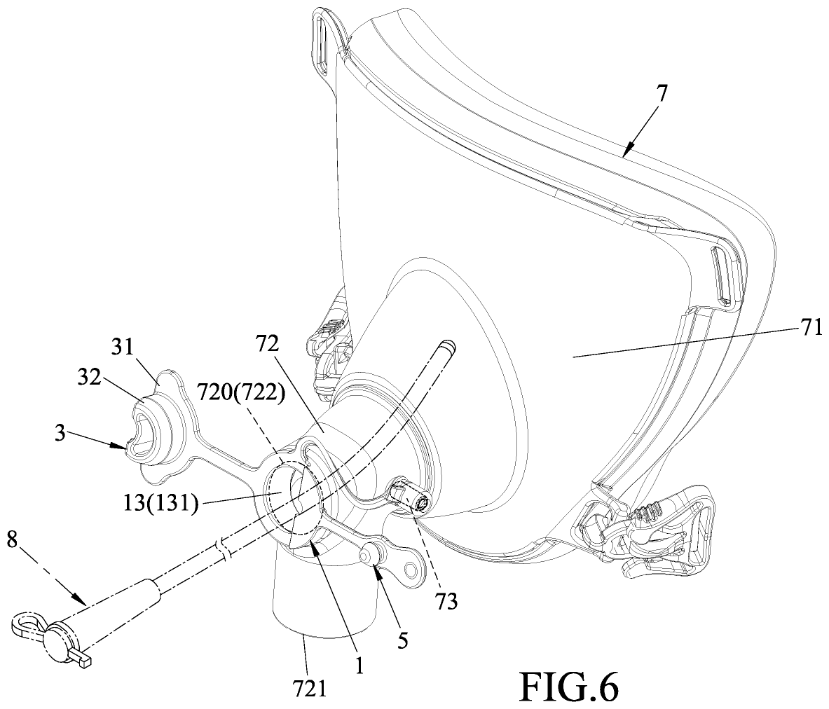

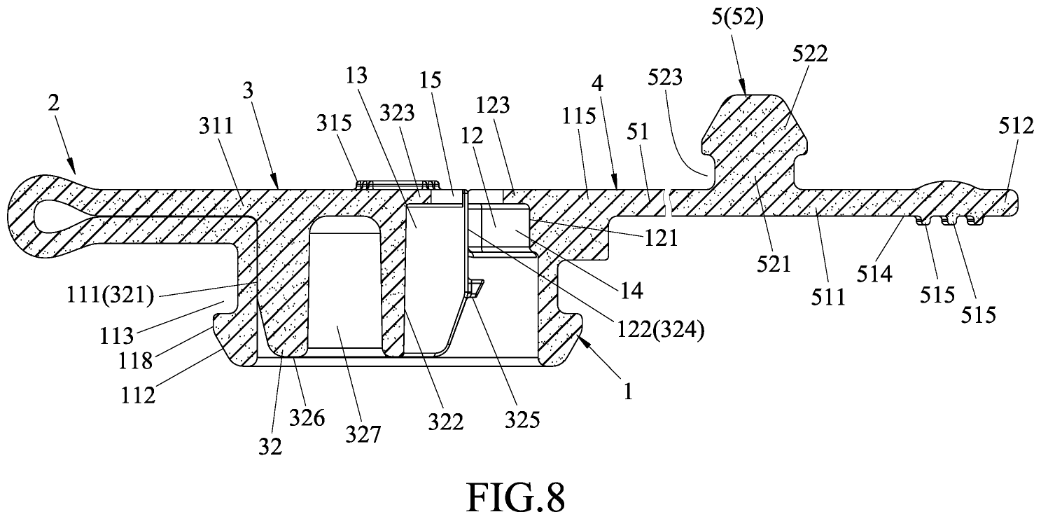

Referring to FIG. 6, in combination with FIGS. 1 and 2, to connect the nasogastric tube connector device of this embodiment to the breathing tube 72 of the respirator mask 7, the connector body 1 is inserted into the through hole 722 of the breathing tube 72 until the hole-defining wall 720 of the through hole 722 is received in the annular groove 113 and the annular protrusion 118 (see FIG. 8) abuts against an inner peripheral edge of the hole-defining wall 720 so as to improve airtightness between the connector body 1 and the breathing tube 72 and to secure the connector body 1 to the breathing tube 72.

It should be noted that in other variations of this embodiment, and under the condition that wearing of the respirator mask 7 by a user is not affected, the through hole 722 may be formed in the mask body 71, but is not limited thereto.

The nasogastric tube connector device of this embodiment is placeable in three states, namely an open state (see FIG. 6), a use state (see FIG. 2), and a closed state (see FIG. 1).

In the open state, as shown in FIG. 6, the first and second plug members 3, 5 are away from the opening 131 of the space 13 to keep the space 13 open. At this time, the nasogastric tube 8 can be inserted smoothly into the breathing tube 72 through the opening 131 of the space 13, and the respirator mask 7 attached with the nasogastric tube connector device of this embodiment can be worn on a face of the user.

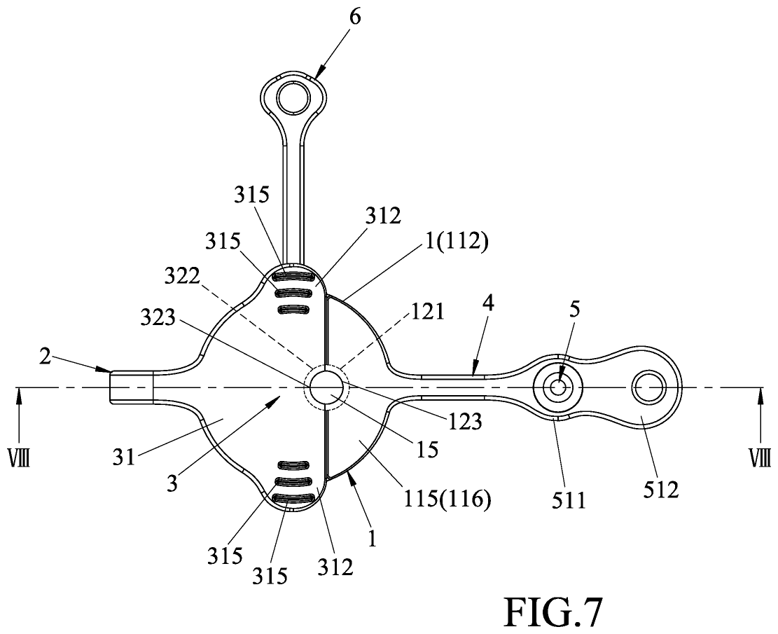

In the use state, as shown in FIGS. 2, 3, 7 and 8, the first plug body 32 is inserted through the opening 131 into the space 13 of the connector body 1. At this time, the convex surface 321 abuts against the inner peripheral surface 111 of the connector body 1, the connecting surfaces 324 respectively abut against the side surfaces 122, the protrusions 325 respectively abut against lower portions of the side surfaces 122, and the first extension portion 31 covers a junction of the upper peripheral surface 114 and the first plug body 32. Further, two opposite ends of the first cover section 311, which are respectively proximate to the connecting surfaces 324, respectively abut against the abutment surfaces 117, and the first holding sections 312 protrude outwardly and respectively from the outer peripheral surface 112 of the connector body 1.

Moreover, the first and second concave surfaces 121, 322 cooperatively define the main tube hole 14, and cooperatively surround the nasogastric tube 8; while the first and second concave flanges 123, 323 cooperatively define the auxiliary tube hole 15. Since the first and second concave flanges 123, 323 are made of elastic silicone rubber, when the nasogastric tube 8 is inserted into the main tube hole 14 through the auxiliary tube hole 15, the first and second concave flanges 123, 323 surround the nasogastric tube 8 in a tight-fitting manner to maintain good airtightness.

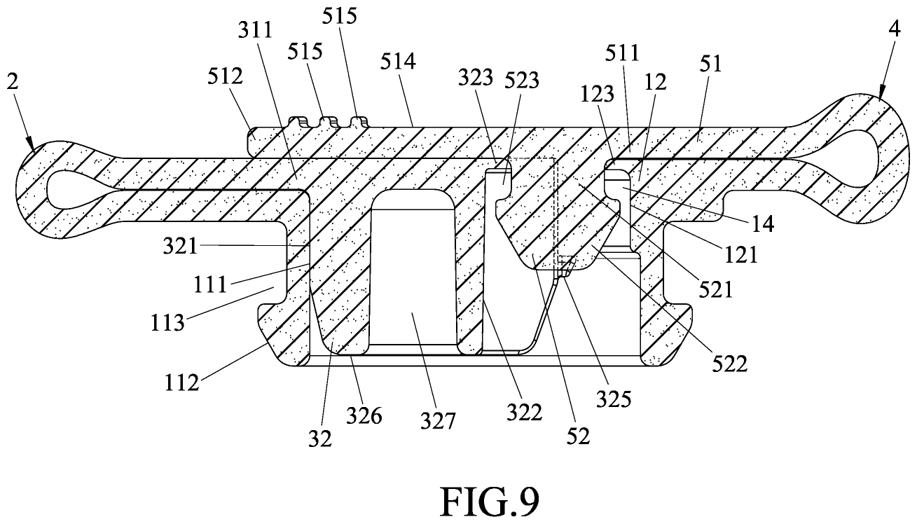

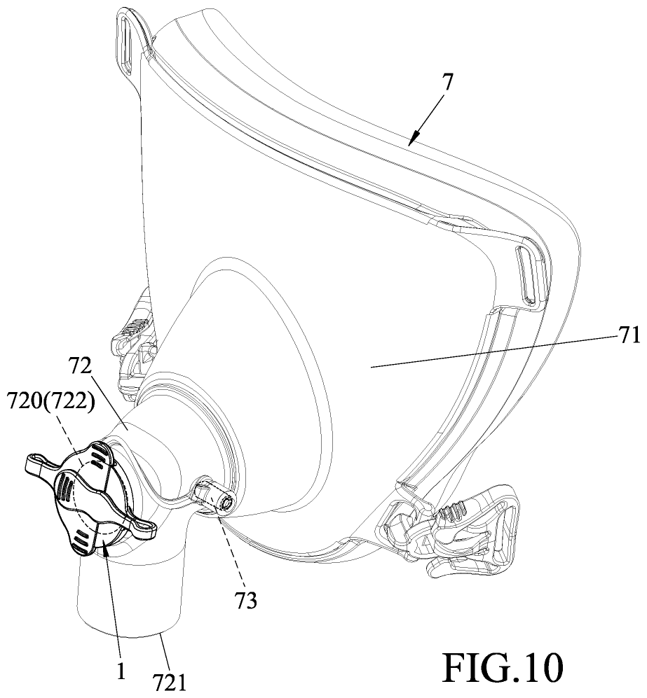

In the closed state, as shown in FIGS. 8 to 10, the first plug body 32 is inserted into the space 13 of the connector body 1, and the second plug body 52 is inserted through the auxiliary tube hole 15 into the main tube hole 14 to sealingly close the opening 131 of the space 13. At this time, the first and second concave flanges 123, 323 are engaged to the annular engaging groove 523, the head portion 522 of the second plug body 52 is located within the space 13, and the second extension portion 51 covers a junction of the neck portion 521 of the second plug body 52, the first concave flange 123, and the second concave flange 323. Through the configuration relationship of the above components, when oxygen is supplied through the breathing tube 72, air in the breathing tube 72 will not escape via the through hole 722 so as to maintain a good positive pressure oxygen supply state between the respirator mask 7 and the face of the user. It should be noted that in this state, the nasogastric tube 8 is not required and is removed.

With reference to FIGS. 2 and 6, when it is desired to remove the respirator mask 7 from the face of the user, it is only necessary to pull one of the first holding sections 312 of the first plug member 3 so as to separate the first plug body 32 from the connector body 1, after which the nasogastric tube 8 is pulled out of the connector body 1 through the opening 131, and finally, the respirator mask 7 can be safely removed from the user's face.

It should be noted that, in other variations of this embodiment, the connector body 1, the first connecting tab 2, the first plug member 3, the second connecting tab 4, the second plug member 5 and the retaining member 6 can be separately produced and then connected to each other, as long the first plug member 3 can be inserted into the space 13 of the connector body 1 and can define the main and auxiliary tube holes 14, 15, and the second plug member 5 can be inserted into the main and auxiliary tube holes 14, 15.

From the foregoing, the advantages of this disclosure can be summarized as follows:

1. When this embodiment is used with the nasogastric tube 8, the nasogastric tube connector device of this embodiment is first placed in the use state, after which the nasogastric tube 8 is inserted into the main tube hole 14, and the first and second concave flanges 123, 323 cooperatively surround the nasogastric tube 8 in a tight-fitting manner to improve airtightness and to maintain positive pressure between the respirator mask 7 and the user's face, so that a good oxygen supply can be effectively provided.

2. The nasogastric tube connector device of this embodiment can be placed in the closed state to sealingly close the opening 131 of the space 13 of the connector body 1. Through this, the respirator mask 7 can be normally used for supplying oxygen without replacing the breathing tube 72, so that this embodiment has the advantage of convenient use.

3. Through the buffer chamber 327 of the first plug body 32, the first plug body 32 can be compressed when squeezed to facilitate insertion of the first plug body 32 into the space 13 through the opening 131.

4. Through the retaining member 6, separation of the oxygen supply tube 73 from the respirator mask 7 can be prevented due to pulling.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A nasogastric tube connector device suitable for connection with a respirator mask having a through hole, said nasogastric tube connector device comprising:
an annular connector body for detachably engaging the through hole, said annular connector body including an inner peripheral surface that defines a space, and a fixed member connected to said inner peripheral surface, said fixed member having a first concave surface, and two side surfaces respectively extending from two opposite ends of said first concave surface toward and connected to said inner peripheral surface, wherein said side surfaces, said first concave surface and said inner peripheral surface cooperatively define an opening of said space for passing of a nasogastric tube therethrough;
a first plug member detachably inserted through said opening into said space and including a second concave surface that cooperates with said first concave surface to define a main tube hole communicating with said space for insertion of the nasogastric tube therethrough; and
a second plug member detachably inserted into said main tube hole to sealingly close said opening of said space.

2. The nasogastric tube connector device as claimed in claim 1, wherein said nasogastric tube connector device is placeable in an open state, in which said first plug member and said second plug member are away from said space to keep said space open; a use state, in which said first plug member is inserted into said space, and said first concave surface and said second concave surface cooperatively define said main tube hole and are configured to cooperatively surround the nasogastric tube when the nasogastric tube is inserted through said main tube hole; and a closed state, in which said first plug member is inserted into said space, and said second plug member is inserted into said main tube hole to sealingly close said opening of said space.

3. The nasogastric tube connector device as claimed in claim 2, further comprising a first connecting tab connected between said annular connector body and said first plug member, and a second connecting tab connected between said annular connector body and said second plug member.

4. The nasogastric tube connector device as claimed in claim 2, wherein said first plug member includes a first plug body detachably inserted into said space and having said second concave surface, said first plug body further having a convex surface that is opposite to said second concave surface and that has a curvature matching that of said inner peripheral surface of said annular connector body, and two connecting surfaces each of which extends from one of two opposite ends of said second concave surface to a respective one of two opposite ends of said convex surface, and wherein, when said first plug body is inserted into said space, said convex surface abuts against said inner peripheral surface of said annular connector body, and said connecting surfaces respectively abut against said side surfaces of said fixed member.

5. The nasogastric tube connector device as claimed in claim 4, wherein said first plug body further has two protrusions respectively protruding from said connecting surfaces, and wherein, when said first plug body is inserted into said space, said protrusions respectively abut against lower portions of said side surfaces of said fixed member.

6. The nasogastric tube connector device as claimed in claim 4, wherein said annular connector body further includes an outer peripheral surface opposite to said inner peripheral surface, and an upper peripheral surface connected between upper ends of said inner peripheral surface and said outer peripheral surface, said first plug member further including a first extension portion extending outwardly from said first plug body, and wherein, when said first plug body is inserted into said space, said first extension portion covers a junction of said upper peripheral surface and said first plug body.

7. The nasogastric tube connector device as claimed in claim 6, wherein said first extension portion has a first cover section connected between said first connecting tab and said first plug body, and at least one first holding section extending outwardly from said first cover section, and wherein, when said first plug body is inserted into said space, said at least one first holding section protrudes outwardly from said outer peripheral surface of said annular connector body.

8. The nasogastric tube connector device as claimed in claim 6, wherein said fixed member further has a first concave flange extending inwardly and radially from an upper end of said first concave surface, said first plug body further having a second concave flange extending inwardly and radially from an upper end of said second concave surface and cooperating with said first concave flange to define an auxiliary tube hole, each of said first concave flange and said second concave flange being made of an elastic material, said auxiliary tube hole communicating with said main tube hole and having a hole diameter smaller than that of said main tube hole, said first concave flange and said second concave flange being configured to surround the nasogastric tube in a tight-fitting manner when the nasogastric tube is inserted into said main tube hole through said auxiliary tube hole.

9. The nasogastric tube connector device as claimed in claim 8, wherein said second plug member includes a second plug body detachably inserted into said main tube hole through said auxiliary tube hole, and a second extension portion extending outwardly from said second plug body, and wherein, when said second plug body is inserted into said main tube hole, said second extension portion covers a junction of said second plug body, said first concave flange and said second concave flange.

10. The nasogastric tube connector device as claimed in claim 1, further comprising a retaining member that includes a connecting portion connected to said annular connector body, and a ring portion connected to said connecting portion opposite to said annular connector body.
